(19) United States
(12) Patent Application Publication
(10) Pub. No.: US 2006/0165776 A1
(43) Pub. Date: Jul. 27, 2006

(54) Antidepressant oral pharmaceutical compositions

(76) Inventor: Ramesh Sesha, Monmouth Junction, NJ (US)

Correspondence Address: RAMESH SESHA, 6201 SHADOW OAKS COURT, Monmouth Junction, NJ, 08852, US

(21) Appl. No.: 11/215,911

(22) Filed: Aug. 31, 2005

(51) Int. Cl. A61K 31/381; A61K 9/48; A61K 9/20

(52) U.S. Cl. 424/451; 424/464; 514/438

(57) ABSTRACT

Novel enteric compositions suitable for oral administration comprising Duloxetine or its pharmaceutical derivatives thereof and methods for preparing such compositions are disclosed. Such compositions contain a core consisting of a Duloxetine or its pharmaceutical derivatives thereof, the said core comprised of a pharmaceutically inert nuclei and the Duloxetine or its pharmaceutical derivatives thereof compressed together, an intermediate and an enteric layer. Duloxetine or its pharmaceutical derivatives thereof may be any pharmaceutically acceptable prodrug, salt, solvate or derivative of Duloxetine. The novel compositions prepared according to the present invention have enhanced stability and bioavailability.

### FIELD OF THE INVENTION

The present invention relates to a pharmaceutically acceptable novel enteric compositions comprising serotonin selective reuptake inhibitors, in particular Duloxetine or its pharmaceutically acceptable derivative thereof for oral administration, and a process for preparing such formulations.

### BACKGROUND OF THE INVENTION

Feelings of intense sadness and despair, mental slowing and loss of concentration, pessimistic worry, lack of pleasure, self-deprecation, and variable agitation clinically characterize major depression. Physical changes also occur include insomnia or hypersomnia; altered eating patterns; decreased energy and libido; and disruption of the normal circadian and ultradian rhythms of activity and many endocrine functions.

At molecular level, the diffuse connections of neurotransmitter serotonin may affect many basic psychological functions such as anxiety mechanisms and the regulation of mood, thoughts, aggression, appetite, sex drive and the sleep/wake cycle. Serotonin is one of the most abundant neurotransmitters, originating in neurons deep in the midline of the brainstem, plays an important role in the regulation of mood and a key role in the treatment of depression.

Psychotropic agents can be placed into four major categories. Antianxiety-sedative agents, antidepressants (mood-elevating agents), antimanic or mood stabilizing drugs and neuroleptic drugs. Of these, antidepressants are used to treat moderate to severe depressive illnesses. They are also used to help in treating the symptoms of severe anxiety, panic attacks and obsessional problems. They may also be used to help people with chronic pain, eating disorders and post-traumatic stress disorder. Yet, the treatment of depression relies on a varied group of antidepressant therapeutic agents, in part because clinical depression is a complex syndrome of widely varying severity. The commonly used antidepressants include tricyclic antidepressants that primarily act by inhibiting norepinephrine & variably serotonin transport into nerve endings, thus leading to sustained facilitation of noradrenergic and perhaps serotonergic function in the brain. The newer classes of antidepressants, the inhibitors of monoamine oxidase, increase the brain concentrations of many amines and are also commonly used.

Diagnosis and treatment of depression have advanced recently, stimulated by serotonin selective reuptake inhibitors (SSRIs), which are both effective antidepressants and also are powerful antianxiety agents. SSRIs inhibit the reuptake of serotonin and, thus, increase the concentration of this neurotransmitter in the central nervous system. The mechanism of action for the SSRIs is believed to be the blocking of the uptake pump action on the presynaptic neuron. This increases the amount of serotonin in the synaptic cleft and at the postsynaptic serotonin receptor site, resulting in greater postsynaptic serotonin stimulation. Most widely prescribed serotonin selective reuptake inhibitors (SSRIs) include citalopram, fluoxetine, zimelidine, sertraline, venlafaxine, fluvoxamine, paroxetine, and the like. Duloxetine is amongst the newer drugs in the class of SSRI inhibitors.

Duloxetine is a selective serotonin and norepinephrine reuptake inhibitor (SSNRI) and its molecular structure is as shown below:

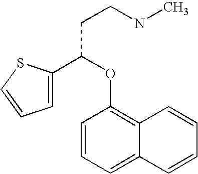

### Duloxetine((S)-N-methyl-3-(1-naphthyloxy)-3-(2-thienyl)propan-1-amine)

Duloxetine is a selective serotonin and norepinephrine reuptake inhibitor (SSNRI) and is available as a white to slightly brownish white solid and is soluble in water. Although the exact mechanisms of the antidepressant and central pain inhibitory action of duloxetine in humans are unknown, the antidepressant and pain inhibitory actions is believed to be because of its potentiation of serotonergic and noradrenergic activity in the CNS. Duloxetine has no significant affinity for dopaminergic, adrenergic, cholinergic or histaminergic receptors in vitro. Duloxetine does not inhibit monoamine oxidase (MAO). Duloxetine undergoes extensive metabolism, but the major circulating metabolites have not been shown to contribute significantly to the pharmacologic activity of duloxetine.

Compounds such as Duloxetine have a dual mechanism of action as they selectively inhibit the uptake of serotonin and norepinephrine. Compounds belonging to the genus class (3-aryloxy-3-substituted propanamines), of which duloxetine is a species have been used for treating a variety of disorders which have been linked to decreased neurotransmission of serotonin and norepinephrine in mammals including obesity, depression, alcoholism, pain, loss of memory, anxiety, smoking, and the like.

Duloxetine is (+)-N-methyl-3-(1-naphthalenyloxy)-2-thiophenepropanamine, and is commonly used as its hydrochloride salt. Eli Lilly markets duloxetine, under the trade name of Cymbalt^{R}, as a delayed release capsule comprising enteric-coated pellets of the drug. It is indicated for the treatment of major depressive disorder and for the treatment of diabetic peripheral neuropathic pain.

Duloxetine is acid labile, and acid hydrolysis of its ether linkage results in a thienyl alcohol and 1-naphthol. 50% of a dosage is hydrolyzed to 1-naphthol within one hour at pH of 1.0, which is achieved under fasting conditions. At a pH of 2.0, 10% of the dosage degrades to 1-Naphthol in one hour and at a pH of 4.0, 10% degradation would take up to 63 hours. The reaction scheme showing the conversion of duloxetine to 1-naphthol and its thienyl derivative is as shown below.

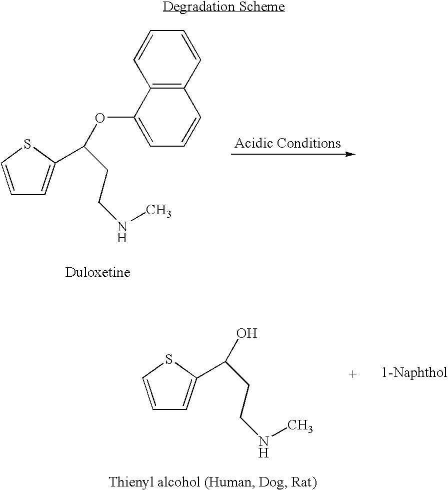

Typically such acid sensitive compounds have been formulated as enteric-coated pellets to protect them from degradation.

Enteric coatings have been used for many years to arrest the release of the drug from orally ingestible dosage forms. Depending upon the composition and/or thickness, the enteric coatings are resistant to stomach acid for required periods of time before they begin to disintegrate and permit slow release of the drug in the lower part of stomach or upper part of the small intestine. Some of the existing art described below disclose different enteric coating formulations:

U.S. Pat. No. 6,897,205 discloses an invention related to a multiparticulate drug form for uniform release of an active ingredient in the small intestine and in the large intestine, comprising at least two forms of pellets A and B having different polymer coatings. The inner polymer coating of pellet form A comprises a methacrylate copolymer whereas the outer polymer coating is an enteric coating, which rapidly dissolves only above pH 5.5, of a methacrylate copolymer which contains acidic groups and has, for example, acrylic acid, but preferably methacrylic acid, residues. The polymer coating of pellet form B comprises a methacrylate copolymer.

Upon oral ingestion the capsule shell dissolves allowing the contents in the capsule to be exposed to the gastric contents. Due to the presence of fluids in the stomach, exposed particles become moistened. If the moist particles do not stick together, they will disperse into the gastric contents and may begin to enter the duodenum based on the size distribution and other factors, which control the gastric transit time. However, if the particles become tacky upon moistening, they may stick together as one or more lumps. In this case, such lumps may behave as large particles and their gastric emptying time will be variable depending upon the size and the strength of the lumps formed. Hence, such a dosage form would not behave as a true multiparticulate system.

U.S. Pat. No. 4,786,505 (Lovgren et al) discloses a pharmaceutical preparation containing omeprazole together with an alkaline reacting compound or an alkaline salt of omeprazole optionally together with an alkaline compound as a core material in a tablet formulation. The core is then enterically coated. The use of the alkaline material, which can be chosen from such substances as the sodium salt of carbonic acid, are used to form a micro-pH around each omeprazole particle to protect omeprazole which is highly sensitive to acid pH. The powder mixture is then formulated into enteric-coated small beads, pellets, tablets and may be loaded into capsules by conventional pharmaceutical procedures.

U.S. Pat. No. 5,837,291 to Shin-etsu Chemical Co., Ltd. discloses a method of preparing an enteric preparation coated with an enteric coating agent without drying, said method comprising applying to a solid dosage form a non-solvent coating composition consisting essentially of a fine powder polymeric enteric coating agent while spraying a liquid plasticizer therefor. It also provides an enteric preparation wherein the solid dosage form is granules of the active ingredient, said liquid plasticizer is triethyl citrate. The '291 patent claims an enteric preparation wherein the particle diameter of said fine powder enteric coating agent is 10 micrometers or less. The enteric coating agent used in the invention is hydroxypropylmethyl cellulose acetate succinate (HPMCAS), because it has a low softening temperature and superior film forming properties.

U.S. Pat. No. 6,224,910 assigned to Bristol-Myers Squibb Co. provides a high drug load enteric coated pharmaceutical composition which includes a core comprised of a medicament which is sensitive to a low pH environment or acid labile drugs such as 2′,3′-dideoxyinosine (ddl or didanosine) pravastatin, erythromycin and the like; which composition is preferably in the form of beadlets having an enteric coating formed of methacrylic acid copolymer, plasticizer and an additional coat comprising an anti-adherent. The so-called beadlets have excellent resistance to disintegration at lower pH but have excellent drug release properties at pH greater than 4.5-5. A novel method of making said pharmaceutical composition is also disclosed.

U.S. Pat. No. 5,225,202 assigned to E.R. Squibb & Sons, Inc. discloses enteric-coated pharmaceutical compositions utilizing neutralized hydroxypropyl methylcellulose phthalate polymer (HPMCP) coating. The pharmaceutical compositions disclosed comprise an acid labile medicament core, a disintegrant, one or more buffering agents to provide added gastric protection in addition to the enteric coating, as well as the enteric coating and a plasticizer. The pharmaceutical composition may also include one or more lactose, sugar or starch fillers.

U.S. Pat. No. 6,224,911 assigned to Syntex LLC, discloses a process for preparing enteric coated pharmaceutical dosage forms, which comprises combining in water anionic polymers, plasticizers, one or more optional excipients, and a volatile base to form an aqueous enteric coating dispersion; and coating an uncoated pharmaceutical dosage form with the aqueous dispersion. Thus, the absorption of a drug as it passes through the alimentary canal can be controlled by enteric coating the pharmaceutical with a substance which will at certain pH values retard release of the drug while at other pH values promote disintegration and/or leaching of the drug from the dosage form. For example, a coat comprised of an anionic polymer such as cellulose acetate phthalate prevents premature disintegration of the formulation in the acidic environment of the stomach and promotes rapid release of the drug in the intestine.

The U.S. Pat. No. 4,377,568 describes a description of aqueous alcoholic enteric coating dispersions. However, organic solvents have to be recycled and can result in contamination of the enteric coat. When water is used to prepare an enteric coating dispersion, a detackifier and glidant (e.g., talc) may be needed to avoid sticking or clumping of the pharmaceutical dosage forms during the application process.

The above literature reports various techniques for enteric dosage forms for acid-labile drugs. However, Duloxetine present a different kind challenge to inventors because of its high instability to acidic conditions. It belongs to the class of 3-aryloxy-3-substituted propanamines, which are potent and non-selective inhibitors of neuronal reuptake of serotonin and norepinephrine (a dual reuptake inhibitor). These dual reuptake inhibitors may have improved efficacy for the treatment of severe depression and is more effective than selective reuptake inhibitors for treatment of pain. Compared to SSRIs, Duloxetine has a shorter onset of action because of its effects on both 5HT and norepinephrine. It is well absorbed after oral administration of capsules containing enteric-coated pellets, with a median time to maximum concentration (Tₘₐₓ) of 6 hours, it is highly protein bound (>90%), and it exhibits a mean plasma elimination half-life of 12.1 hours. Duloxetine is metabolized to several inactive metabolites in the liver.

The U.S. Pat. No. 5,023,269 patent claims compounds belonging to this class and the compound duloxetine has also been claimed in this patent. The invention provides pharmaceutical formulations comprising a compound of the above formula and a pharmaceutically acceptable carrier, diluent or excipient thereof. The '269 patent provides an example of a suspension of duloxetine succinate (example 7 of the '269 patent) with sodium carboxymethylcellulose as a suspending agent. The other excipients in the suspension include syrup base, benzoic acid solution, flavor, color and water.

U.S. Pat. No. 5,508,276 assigned to Eli Lilly discusses duloxetine, in the form of enteric pellets of which the enteric layer comprises hydroxypropylmethylcellulose acetate succinate.

The invention of the '276 patent was provided with a superior enteric formulation of duloxetine, by using hydroxypropylmethylcellulose acetate succinate as the enteric-coating polymer. The enteric dosage forms have been employed because it is very important that this drug should not be exposed to gastric acid prior to absorption. Although it is stable at alkaline pH, it gets destroyed rapidly as pH falls. Therefore, if the micro encapsulation or the enteric coating is disrupted (e.g., by trituration of the compound or chewing the capsule), the drug would be exposed to degradation by the gastric acid in the stomach. Duloxetine was also found to react with many enteric coatings to form a slowly- or even insoluble coating. Because of this unexpected cross-reactivity, formulations in pellet form were found to have a disadvantageous drug-releasing profile and low bioavailability. Thus the instability of Duloxetine at acidic pH is a known problem that has been addressed in a way by the capsule dosage form containing enteric coated Duloxetine pellets with hydroxypropylmethylcellulose acetate succinate as the enteric-coating polymer.

However, according to one literature report, Duloxetine has been found to react with polymer degradation products or residual free acids present in the enteric polymers hydroxypropyl methyl cellulose acetate succinate (HPMCAS) and hydroxypropyl methyl cellulose phthalate (HPMCP) in dosage formulations to form succinamide and phthalamide impurities respectively. The rate of formation of these impurities is accelerated by heat and humidity. As mentioned above, Duloxetine is unstable in solution at pH values less than 2.5, enteric polymer-coated formulations have been developed to prevent its acid degradation in the stomach and to provide for subsequent rapid disintegration and release in the duodenum.

During the course of development of the HPMCAS coated pellet formulation, Duloxetine succinamide that eluted after Duloxetine was detected by HPLC analysis of samples stressed at 60° C. for 14 days. This impurity was also detected in stability samples stored at 30° C./60% relative humidity and 40° C./75% relative humidity. Subsequent analysis of stability samples of HPMCP coated tablets indicated the presence of Duloxetine phthalamide that also eluted after Duloxetine.

These impurities were the result of reaction between enteric polymer substituents and drugs containing nucleophilic functional groups, i.e. reaction of Duloxetine with phthaloyl and succinoyl moieties present in the enteric polymers HPMCP and HPMCAS, respectively. Because the enteric polymers are physically separated from Duloxetine by a sub-coating, the formation of these impurities indicate the migration of either Duloxetine or the phthaloyl or succinyl moieties through the subcoating to enable physical contact and reaction.

Therefore, as discussed above, duloxetine is prone for degradation at lower pH that normally prevail in stomach and such a degradation results in 1-Naphthol impurity, which is known to be very toxic and cause several side effects, the stability of duloxetine in formulation is therefore a key challenge. Hence, there is a need for stabilized formulation comprising duloxetine or its derivative that is free from 1-Naphthol. i.e. an oral stable dosage form comprising duloxetine or its derivative manufactured by an expedient manufacturing process to yield a composition which is clinically superior and provides greater choice for both prescriber and patient.

To this end, the present invention discloses a simple, stable, enteric oral pharmaceutical composition for treatment of depression and other related psychotropic disorders. The said composition comprises duloxetine or its pharmaceutically acceptable derivative in a formulation that doesn't comprise either any alkaline reacting compound or other buffering agents. Particularly, the composition is made by simple manufacturing process and thus brings the advantage of simple composition with easy process.

### SUMMARY OF THE INVENTION

The present invention provides novel enteric compositions suitable for oral administration comprising: (a) a core comprising Duloxetine or its pharmaceutically acceptable derivative thereof. The said core comprised of pharmaceutically inert nuclei and the Duloxetine or its pharmaceutically acceptable derivative thereof mixed and compressed together, (b) an intermediate layer, and (c) an enteric layer; such that (the said composition is substantially free of any alkaline reacting compounds. The said novel enteric composition is manufactured by constituting a core which in turn is formed of nuclei and said duloxetine or its pharmaceutically acceptable derivative thereof mixed together and then compressed together followed by an intermediate layer followed by an enteric layer.

Accordingly, it is an object of the present invention to provide a pharmaceutical composition and a process of manufacturing the same for the delayed release of antidepressant like duloxetine or its pharmaceutically acceptable derivative thereof.

Another object of the present invention is an enteric released solid pharmaceutical composition adapted for oral administration.

Yet another object of the present invention is an enteric duloxetine composition comprised of duloxetine or its derivative thereof in a core formed by nuclei coated with an intermediate layer followed by an enteric layer.

The present invention therefore provides core comprised of said nuclei and duloxetine or its pharmaceutically acceptable derivative thereof are mixed together, granulated and then compressed together with an intermediate layer followed by an enteric layer.

According to one embodiment of the invention, the said pharmaceutically inert nuclei contain one or more pharmaceutically acceptable materials.

The present invention therefore comprises, other pharmaceutically acceptable excipients as may be present additionally with the said pharmaceutically inert nuclei and Duloxetine or its pharmaceutically acceptable derivative thereof.

In yet another embodiment of the invention, a pharmaceutically acceptable lubricant may be present additionally with the said pharmaceutically inert nuclei and Duloxetine or its pharmaceutically acceptable derivative thereof.

In another embodiment of the invention, the pharmaceutically inert nuclei, has a particle size preferably in the range of about 100 μm and about 500 μm.

Yet another object of the present invention is that the invention relates to the intermediate layer as comprised of at least one or more water-soluble layers comprising non-acid inert pharmaceutical excipients.

In another embodiment of the invention, the intermediate layer may comprise of at least one organic or inorganic polymer or a mixture thereof.

In another embodiment of the invention, the enteric layer contains at least one enteric polymer.

Yet another object of the present invention is the process of manufacturing the oral solid enteric or delayed release pharmaceutical composition. In other words, the invention provides a delayed or enteric release, solid pharmaceutical composition comprising at least one antidepressant pharmaceutically active agent, particularly, duloxetine or its pharmaceutically acceptable derivatives thereof manufactured by the process comprising the steps of:

(i) mixing the pharmaceutically inert nuclei with the Duloxetine or its derivative thereof,

(ii) granulating and compressing the product of step (i) to form a core comprising Duloxetine or its derivative thereof,

(iii) coating the said core obtained in step (ii) with an intermediate layer, and

(iv) coating a product of step (iii) with an enteric layer.

In one embodiment of the invention, the said step (i) is carried out by spraying a medium containing a Duloxetine or its pharmaceutically acceptable derivative thereof onto pharmaceutically inert nuclei in a fluidized bed granulator, followed by drying the product thus obtained.

In a further embodiment of the invention, the instant process additionally comprises mixing of pharmaceutically inert nuclei or the product of step (i) with at least one pharmaceutical excipient.

In a still further embodiment of the invention, the instant process additionally comprises mixing of pharmaceutically inert nuclei or the product of step (i) with pharmaceutical excipient, preferably with at least one lubricant.

In yet another embodiment of the invention, the intermediate and/or the layer is applied using spray coating.

The present invention provides obvious benefits being simple and fast operational process for manufacturing said oral solid enteric release pharmaceutical composition.

Further aspects and embodiments of the invention may become apparent to those skilled in the art from a review of the following detailed description, taken in conjunction with the examples and the claims. It must be understood that that the present disclosure is intended as illustrative, and is not intended to limit the invention to the specific embodiments described herein.

### DETAILED DESCRIPTION OF THE INVENTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a”, “an” and “the” include plural reference unless the context clearly dictates otherwise. Unless defined otherwise, all technical and scientific terms used herein have the same meaning as commonly understood to one of ordinary skill in the art to which this invention belongs.

Unless defined otherwise, all technical and scientific terms used herein have the same meaning as commonly understood to one of ordinary skill in the art to which the invention belongs. Although any methods, devices and materials similar or equivalent to those described herein can be used in the practice or testing of the invention, the preffered methods, devices and materials are now described.

All publications mentioned herein are incorporated herein by reference for the purpose of describing and disclosing the subject components of the invention that are described in the publications, which components might be used in connection with the presently described invention.

The information below is not admitted to be prior art to the present invention, but is provided solely to assist the understanding of the reader.

The details of one or more embodiments of the invention are set forth in the description and the examples below. Other features, objects, and advantages of the invention will be apparent from the description and from the claims.

The present invention provides novel enteric compositions suitable for oral administration comprising: (a) a core comprising therapeutically effective amount of Duloxetine or pharmaceutically acceptable derivative thereof, the said core comprised of pharmaceutically inert nuclei and the Duloxetine or pharmaceutically acceptable derivative thereof mixed and compressed together, (b) an intermediate layer, and (c) an enteric layer; with a provision that the said Duloxetine or pharmaceutically acceptable derivative thereof is substantially free of any alkaline reacting compound.

The term “pharmaceutically acceptable derivative” means various pharmaceutical equivalent isomers, enantiomers, complexes, salts, hydrates, polymorphs, esters etc of duloxetine.

The term composition includes but not limited to solutions and/or suspensions, dispersions, concentrates, ready mix, powders, granules, tablets, micro-tablets, capsules, pellets, comprising duloxetine or its pharmaceutically acceptable derivative thereof in a core constituted by nuclei and coated with intermediate layer/s followed by enteric layer.

The term “therapeutically effective amount” means an amount of the drug which is capable of eliciting a physiological response in a human patient. More specifically, the term “therapeutically effective amount” means the amount of drug, which is capable of treating depression and related psychotropic disorders.

The said medicament according to the present invention comprises a formulation substantially as herein described, and in particular a capsule or a tablet or micro-tablets or granules or pellets filled in capsule formulaton, typically an enteric or delayed release capsule formulation substantially as hereinafter further described.

Suitably a formulation according to the present invention provides a novel enteric dosage form, preferably capsules or micro-tablets in capsule form comprising core comprising pharmaceutically inert nuclei which is comprised of duloxetine or its pharmaceutically acceptable derivative thereof and optionally one or more suitable excipients and the said core coated with intermediate layer/s followed by enteric layer and the process for preparing the same.

In a preferred embodiment of the present invention, the pharmaceutically inert nuclei, as the name may suggest, is composed of a substance or a mixture of such substances that are pharmaceutically inert and preferably do not interfere with the biological action of the Duloxetine or its pharmaceutically acceptable derivative thereof. Preferable examples of such substances include lactose, dextrose, saccharose, starch and other sugars. A wide variety of other substances can also be used in developing the inert nuclei. If desired, the inert nuclei may also contain other pharmaceutically acceptable excipients with the exception of any alkaline reacting substances. Particle size of the pharmaceutically inert nuclei in the absence of the Duloxetine or its pharmaceutically acceptable derivative thereof is preferably in the range of about 100 μm to about 500 μm. It is an advantageous feature of the present invention that a particle size outside this preferable range can also be used, if desired.

The said core is comprised of pharmaceutically inert nuclei and the Duloxetine or its pharmaceutically acceptable derivative thereof mixed and compressed together. The mixing here may be purely physical mixing, deposition, coating, adsorption, aggregation or adhesion and alike. Such a mixing of the said pharmaceutically inert nuclei and Duloxetine or its pharmaceutically acceptable derivative thereof may be achieved in several different ways, including those already documented in the prior art. According to one of the embodiment of the invention, such a mixing is achieved by granulation, and preferably through fluidized bed granulation. A typical fluidized granulation would involve fluidizing the pharmaceutically inert nuclei with the inlet air and spraying the (binder) solution of the Duloxetine or its pharmaceutically acceptable derivative thereof on the fluidized bed. Such a process results in formation of granules comprising pharmaceutically inert nuclei and Duloxetine or its pharmaceutically acceptable derivative thereof, which are then compressed together. It is also possible that during this process, the Duloxetine or its pharmaceutically acceptable derivative thereof may be present onto the pharmaceutically inert nuclei and the contents as such are compressed. Yet another possibility includes partial or complete agglomerization of the Duloxetine or its pharmaceutically acceptable derivative thereof and pharmaceutically inert nuclei, which are then compressed together. In another embodiment of the invention, the said core may be prepared by contacting pharmaceutically inert nuclei with a medium containing the Duloxetine or its pharmaceutically acceptable derivative thereof, drying the product and subjecting the product to compression. In yet another preferred embodiment of the invention, such a medium containing the Duloxetine is an aqueous medium. Yet another way of preparing the said core could be by coating of the pharmaceutically inert nuclei with Duloxetine or its pharmaceutically acceptable derivative thereof using one of the coating techniques.

In general, the present invention provides a process for the manufacture of a pharmaceutical product. The process comprises core comprised of duloxetine or its pharmaceutically acceptable derivative thereof with pharmaceutically inert substance (nuclei) and the said core coated with intermediate layer/s followed by coating with enteric polymer/s. The said core is prepared by mixing of duloxetine or its pharmaceutically acceptable derivative thereof with pharmaceutically inert nuclei and optionally mixed with other suitable pharmaceutical excipients. The said core is then coated with intermediate layer/s followed by enteric layer.

Hence, in a preferred embodiment of the present invention, the novel enteric compositions of the present invention comprise at least one intermediate layer that separates the core comprising duloxetine or its pharmaceutically acceptable derivative thereof and the enteric layer. The intermediate layer is preferably composed of a substance (or a mixture of such substances) that do not react or affect the stability of the core comprising duloxetine or its pharmaceutically acceptable derivative thereof nor adversely affect bioavailability or release of the Duloxetine or its pharmaceutically acceptable derivative thereof. Typical examples of such substances that can be used in the intermediate layer include organic or inorganic polymers, sugars and alike. In one embodiment of the invention, the intermediate layer comprises at least one substance selected from a group comprising of silicon dioxide, titanium dioxide, silica, talc, microcrystalline cellule, sodium lauryl sulphate, sodium steryl fumarate, polyethylene glycol, polyvinylpyrrolidinone, polyvinyl alcohol, hydroxypropylcellulose and hydroxymethylcellulose. It is an advantageous feature of this invention that the intermediate layer may also contain other pharmaceutically acceptable excipient if desired. The intermediate layer may be applied to the core using any known technique. These techniques include with any limitation for example powder coating, spraying, pan coating and alike.

In one embodiment of the invention, the enteric layer is comprised of a material that is stable in acidic medium of the stomach and thereby avoids the direct interaction between the acid medium and the contents of the composition. It is preferable that the enteric coat comprises at least one enteric polymer. Preferable examples of such enteric polymers without any limitation include, polyvinyl acetyl phthalate (PVAP) and devatives thereof, vinyl copolymers, hydroxypropylmethylcellulose (HPMC), methacrylate copolymers and acrylic acid polymers and derivatives thereof and alike. Several other commercially available enteric polymers can also be used as enteric coat if desired which include without any limitation Eudragit (Rohm Pharma), Aquateric (FMC Corporation), and alike. It is an advantageous feature of the invention that the enteric layer may also contain other pharmaceutically acceptable excipient such as talc, pigments, colouring agents, flavouring agents, stabilizers, binders, lubricants and alike if desired. The enteric layer may be applied using known techniques including for example those described for intermediate layer.

The novel compositions according to the present invention are substantially free of any alkaline reacting compound. The meaning of the expression “substantially free of any alkaline reacting compound” should be taken herein to mean a composition that does not contain substantial amount of any alkaline reacting compound or a composition in which the amount of alkaline reacting compound is not sufficient to setup an alkaline micro environment around the active principle when it is in contact with an acid or neutral medium.

The novel compositions according to the present invention may optionally contain other pharmaceutically acceptable excipient such as diluents, binders, plastifiers, fillers, surfactants, pigments, stabilizers, disintegrating agents, lubricants, wetting agents, colouring agents, flavouring agents and alike.

The novel compositions of the present invention can be provided in several forms that are suitable for oral administration. In one preferred embodiment, the composition according to the present invention is provided in a tablet form. In another preferred embodiment, such composition is in the form of micro-tablets in capsule e.g. a gelatine capsule.

The novel enteric compositions according to the present invention generally comprise a core representing about 5 to about 98% by weight based on the total weight of the composition, rest being accounted by an intermediate and the enteric layer.

The present invention further comprises a process of preparing a pharmaceutical product, or a pharmaceutical formulation, or a medicament substantially as hereinbefore described. Suitably such a process comprises providing at least one antidepressant such as duloxetine or its pharmaceutically acceptable derivative thereof in a core constituted by mixing duloxetine or its pharmaceutically acceptable derivative thereof with a pharmaceutically inert nuclei optionally with other suitable pharmaceutical excipients and granulated optionally and the said core is further coated with one or more non-acid inert pharmaceutical excipients as “intermediate layer/s” followed by coating with at least one enteric polymer. The said process yields a novel enteric pharmaceutical formulation, typically in micro-tablets or tablets or granules/pellets in capsules (to be filled in capsules) form.

The present invention will now be further illustrated with reference to the following examples, which does not limit the scope of the invention in any way. Further different strengths of the formulation may be achieved by proportionately using a dose weight scale up or scale down formula. The concentration of the excipients may also be varied or modified to achieve the desired dissolution profile by a skilled artisan.

### EXAMPLES

### Example 1

An enteric-composition of Duloxetine (equivalent to 20 mg base) according to present invention was prepared as follows.

### 20 mg Duloxetine Base/Capsule

| | |
|---|---|
| | mg/capsule |
| mg/microtablet | (×10 microtablets) |

Core

| | | |
|---|---|---|
| Duloxetine (EQ base) | 2 | 20 |
| HPMC | 1 | 10 |
| Lactose | 19 | 190 |
| Hydrogenated castor oil | 0.24 | 2.4 |
| Crospovidone | 1.2 | 12 |
| Water | qs | qs |

Intermediate layer

| | | |
|---|---|---|
| Talc | 0.6 | 6 |
| Titanium dioxide | 0.24 | 2.4 |
| HPMC | 1.2 | 12 |
| Water | qs | qs |

Enteric layer

| | | |
|---|---|---|
| Methacrylic acid copolymer | 2.2 | 22 |
| Triethylcitrate | 0.33 | 3.3 |
| Talc | 0.44 | 4.4 |
| Water | qs | qs |

Procedure:

Lactose nuclei having a particle size of about 250 μm were prepared according to the known methods. Appropriate quantity of Hydroxypropylmethyl cellulose (HPMC) and Duloxetine hydrochloride were dissolved in water and the contents were homogenized. The homogenized suspension was then slowly sprayed onto the lactose nuclei in a fluidised bed granulator. After all the suspension was sprayed, the nuclei were dried and mixed with hydrogenated castor oil and crospovidone. The mixed contents are then compressed to obtain microtablets (diameter of about 3 mm). These microtablets were then coated with an intermediate layer having composition as mentioned above. Typically, the intermediate layer was prepared by dissolving HPMC in water followed by addition of talc and titanium dioxide. The resulting contents after homogenisation were sprayed onto the prepared microtablets using a suitable coating device. Finally, these intermediate layer coated microtablets were coated with the enteric layer having composition as mentioned above. The enteric layer was prepared by mixing aqueous solutions of triethyl citrate, methacrylic acid copolymer (Eudragit) and talc and sprayed on microtablets already coated with intermediate layer. These microtablets were filled in gelatin capsules and analysed for stability upon storage.

### Example 2

An enteric-composition comprising Duloxetine (equivalent to 30 mg base) according to present invention was prepared as described above in Example I. The composition was prepared in the form of micro-tablets filled in the gelatin capsule.

### 30 mg Duloxetine Base/Capsule

| | |
|---|---|
| | mg/capsule |
| mg/microtablet | (×20 microtablets) |

Core

| | | |
|---|---|---|
| Duloxetine (EQ base) | 1.5 | 30 |
| HPMC | 0.75 | 15 |
| Lactose | 8.7 | 174 |
| Hydrogenated castor oil | 0.131 | 2.62 |
| Crospovidone | 2.05 | 41 |
| Water | qs | qs |

Intermediate layer

| | | |
|---|---|---|
| Talc | 0.375 | 7.5 |
| Titanium dioxide | 0.15 | 3 |
| HPMC | 0.75 | 15 |
| Water | qs | qs |

Enteric layer

| | | |
|---|---|---|
| Methacrylic acid copolymer | 1.35 | 27 |
| Triethylcitrate | 0.20 | 4 |
| Talc | 0.28 | 5.6 |
| Water | qs | qs |

Procedure: Same as described in Example-1

### Examples 3

An enteric-composition comprising Duloxetine (equivalent to 30 mg base) according to present invention was prepared as follows. The composition was prepared in the form of micro-tablets filled in the gelatin capsule.

### 30 mg Duloxetine Base/Capsule

| | |
|---|---|
| | mg/capsule |
| mg/microtablet | (×20 microtablets) |

Core

| | | |
|---|---|---|
| Duloxetine (EQ base) | 1.5 | 30 |
| HPMC | 1.5 | 30 |
| Lactose | 8.7 | 174 |
| polyethylene glycol 6000 | 0.157 | 3.14 |
| Polysorbate-80 | 0.04 | 0.8 |
| Crospovidone | 2.05 | 41 |
| Water | qs | qs |

Intermediate layer

| | | |
|---|---|---|
| Talc | 0.375 | 7.5 |
| Titanium dioxide | 0.15 | 3 |
| HPMC | 0.75 | 15 |
| Water | qs | qs |

Enteric layer

| | | |
|---|---|---|
| CAP | 1.4 | 28 |
| Triethylcitrate | 0.22 | 4.4 |
| Talc | 0.30 | 6 |
| Water | qs | qs |

Procedure: Same as described in Example-1

### Examples 4

An enteric-composition comprising Duloxetine (equivalent to 30 mg base) according to present invention was prepared as follows. The composition was prepared in the form of micro-tablets filled in the gelatin capsule.

### 30 mg Duloxetine Base/Capsule

| | |
|---|---|
| | mg/capsule |
| mg/microtablet | (×20 microtablets) |

Core

| | | |
|---|---|---|
| Duloxetine (EQ base) | 1.5 | 30 |
| HPMC | 1.5 | 30 |
| Lactose | 8.7 | 174 |
| Hydrogenated castor oil | 0.157 | 3.14 |
| Polysorbate | 0.04 | 0.8 |
| Crosscarmellose sodium | 2.05 | 41 |
| Water | 8.25 | 165 |

Intermediate layer

| | | |
|---|---|---|
| Talc | 0.4 | 8 |
| Titanium dioxide | 0.15 | 3 |
| HPMC | 0.75 | 15 |
| Water | 5 | 100 |

Enteric layer

| | | |
|---|---|---|
| Methacrylate copolymer | 1.4 | 28 |
| Triethylcitrate | 0.22 | 4.4 |
| Talc | 0.30 | 6 |
| Water | 5.82 | 116.4 |

Procedure: Same as described in Example-1

In another set of examples, the compositions described in Examples 1 to 4 were compressed into conventional tablets and then coated with the respective intermediate and enteric layers.

1. An oral pharmaceutical composition comprising:
(a) a core comprising duloxetine or its pharmaceutically acceptable derivative thereof and the said core comprised of pharmaceutically inert nuclei and duloxetine or its pharmaceutically acceptable derivative thereof mixed and compressed together,
(b) an intermediate layer comprising one or more polymers and
(c) an enteric layer comprising one or more enteric polymers;
wherein the said composition is free of alkaline reacting compounds.

2. A pharmaceutical composition of claim 1, wherein the said core comprises of duloxetine or it's pharmaceutically acceptable derivative thereof and pharmaceutically inert nuclei mixed and compressed together.

3. A pharmaceutical composition of claim 3, wherein the said pharmaceutically inert nuclei comprises at least one pharmaceutically acceptable excipient.

4. A pharmaceutical composition of claim 4, wherein the said pharmaceutically inert nuclei is selected from a group comprising lactose, dextrose, saccharose, starch and the like.

5. A pharmaceutical composition of claim 1, wherein the said pharmaceutically inert nuclei have a particle size in the range of about 100 μm to about 500 μm in absence of duloxetine or its pharmaceutically acceptable derivative thereof

6. A pharmaceutical composition of claim 1, wherein the formulation is an oral solid dosage form

7. A pharmaceutical composition of claim 7, wherein the formulation is a capsule, tablet, granules, pill, pellets, spheroids, granules in capsule, pellets in capsule, micro-tablets in capsule or combinations thereof.

8. A pharmaceutical composition of claim 7, wherein the formulation is tablet or capsule or micro-tablets in capsule.

9. The pharmaceutical formulation of claim 1, wherein the tablets or capsule or micro-tablets in capsule comprises enteric released duloxetine or its pharmaceutically acceptable derivatives thereof with pharmaceutically inert nuclei mixed and compressed together and coated with intermediate layer followed by coating with enteric layer.

10. The pharmaceutical formulation of claim 1, wherein the formulation is manufactured comprising the steps of:
(i) mixing pharmaceutically inert nuclei with duloxetine or its pharmaceutically acceptable derivatives thereof;
(ii) compressing the product of step (i) to form a core comprising duloxetine
(iii) coating the said core with an intermediate layer comprising at least one polymer followed by
(iv) coating with one or more enteric polymers.

11. The pharmaceutical formulation of claim 1, wherein the core is manufactured by spraying a solution of duloxetine or its pharmaceutically acceptable derivatives thereof onto pharmaceutically inert nuclei, drying the resultant product and compressing the dried product to form the core comprising duloxetine.

12. The pharmaceutical formulation of claim 1, wherein at least one pharmaceutically acceptable lubricant is present additionally with the said pharmaceutically inert nuclei and Duloxetine or its pharmaceutically acceptable derivatives thereof

13. The pharmaceutical formulation of claim 13, wherein the said lubricant is selected from the group comprising light mineral oil, polyethylene glycol and derivatives thereof, glyceryl behenate, hydrogenated vegetable oil, sodium steryl fumarate calcium silicate and the like.

14. The pharmaceutical formulation of claim 1, wherein the said intermediate layer contains silicon dioxide.

15. The pharmaceutical formulation of claim 1, wherein the said intermediate layer contains at least one organic or inorganic polymer or a mixture thereof

16. The pharmaceutical formulation of claim 1, wherein the said intermediate layer comprises at least one material selected from a group comprising of silicon dioxide, titanium dioxide, talc, sugar and derivatives thereof, polyethylene glycol and derivatives thereof, polyvinylpyrrolidone, polyvinyl alcohol and derivatives thereof, hydroxypropylcellulose, hydroxymethylcellulose, sodium lauryl sulphate, microcrystalline cellulose, colloidal silica, sodium steryl fumarate, starch and derivatives thereof and the like.

17. The pharmaceutical formulation of claim 1, wherein the said enteric layer contains at least one polymer.

18. The pharmaceutical formulation of claim 18, wherein the said enteric layer contains at least one polymer selected from a group comprising of cellulose acetate phthalate (CAP), polyvinyl acetyl phthalate (PVAP), vinyl copolymers, acrylic acid and copolymers and derivatives thereof and the like.

19. A process for manufacture of enteric released formulation of duloxetine or its pharmaceutically acceptable derivatives thereof, the process comprises the steps of: (a) spraying a medium containing duloxetine or its pharmaceutically acceptable derivatives thereof onto the pharmaceutically inert nuclei in a fluidised bed processor followed by drying the resultant product and lubricating the same using suitable lubricant (b) compressing the product of step (a) to form a core containing duloxetine or its pharmaceutically acceptable derivatives thereof (c) coating the said core with an intermediate layer comprising at least one polymer (d) coating the resultant product of step (c) with an enteric layer.

20. A pharmaceutical formulation n solid dosage form prepared by process of claim 20, wherein the said formulation comprises spraying a medium such as water or hydroalcoholic or mixture of one or more organic solvents containing duloxetine or its pharmaceutically acceptable derivatives thereof onto the pharmaceutically inert nuclei such as lactose in a fluidised bed processor followed by drying the resultant product and lubricating the same using suitable lubricant such as hydrogenated castor oil or polyethylene glycol 6000 (b) compressing the product of step (a) to form a core containing duloxetine or its pharmaceutically acceptable derivatives thereof (c) coating the said core with an intermediate layer comprising at least one polymer such as hydroxypropyl methylcellulose (d) coating the resultant product of step (c) with an enteric layer such as acrylic acid polymers like Eudragit^{R}.

21. A pharmaceutical formulation in solid dosage form prepared by process of claim 21, wherein the resultant product is filled in capsules using suitable capsule filling machine.

22. A process of claim 20, wherein the said core comprises of Duloxetine or it's pharmaceutically acceptable derivative thereof and pharmaceutically inert nuclei mixed and compressed together.

23. A process of claim 20, wherein the said pharmaceutically inert nuclei comprises at least one pharmaceutically acceptable excipient.

24. A process of claim 24, wherein the said pharmaceutically inert nuclei is selected from a group comprising lactose, dextrose, saccharose, starch and the like.

25. A process of claim 20, wherein the said pharmaceutically inert nuclei have a particle size in the range of about 100 μm to about 500 μm in absence of duloxetine or its pharmaceutically acceptable derivative thereof

26. A process of claim 20, wherein the formulation is an oral solid dosage form

27. A process of claim 27, wherein the formulation is a capsule, tablet, granules, pill, pellets, spheroids, granules in capsule, pellets in capsule, micro-tablets in capsule or combinations thereof.

28. A process of claim 27, wherein the formulation is tablet or capsule or micro-tablets in capsule.

29. A process of claim 20, wherein the tablets or capsule or micro-tablets in capsule comprises enteric released duloxetine or its pharmaceutically acceptable derivatives thereof with pharmaceutically inert nuclei mixed and compressed together and coated with intermediate layer followed by coating with enteric layer.

30. A process of claim 20, wherein at least one pharmaceutically acceptable lubricant is present additionally with the said pharmaceutically inert nuclei and Duloxetine or its pharmaceutically acceptable derivatives thereof

31. A process of claim 20, wherein the said lubricant is selected from the group comprising light mineral oil, polyethylene glycol and derivatives thereof, glyceryl behenate, hydrogenated vegetable oil, sodium steryl fumarate calcium silicate and the like.

32. A process of claim 20, wherein the said intermediate layer contains silicon dioxide.

33. A process of claim 20, wherein the said intermediate layer contains at least one organic or inorganic polymer or a mixture thereof

34. A process of claim 33, wherein the said intermediate layer comprises at least one material selected from a group comprising of silicon dioxide, titanium dioxide, talc, sugar and derivatives thereof, polyethylene glycol and derivatives thereof, polyvinylpyrrolidone, polyvinyl alcohol and derivatives thereof, hydroxypropylcellulose, hydroxymethylcellulose, sodium lauryl sulphate, microcrystalline cellulose, colloidal silica, sodium steryl fumarate, starch and derivatives thereof and the like.

35. A process of claim 20, wherein the said enteric layer contains at least one polymer.

36. A process of claim 36, wherein the said enteric layer contains at least one polymer selected from a group comprising of cellulose acetate phthalate (CAP), polyvinyl acetyl phthalate (PVAP), vinyl copolymers, acrylic acid and copolymers and derivatives thereof and the like.

37. A method of treating depression and related disorders in a subject in need of treatment, which method comprises administering to the subject a pharmaceutical formulation of claim 1.

38. Use of the pharmaceutical formulation of claim 1 in the manufacture of a medicament for inhibiting serotonin uptake in mammals.

39. Use of the pharmaceutical formulation of claim 1 in the manufacture of a medicament for the treatment of diabetic peripheral neuropathic pain.
